(19) United States
(12) Patent Application Publication
(10) Pub. No.: US 2024/0197704 A1
(43) Pub. Date: Jun. 20, 2024

(54) TREATMENT METHODS AND COMPOSITIONS COMPRISING PERAMPANEL

(71) Applicant: Johns Hopkins University, Baltimore, MD (US)

(72) Inventor: Shipla D. KADAM, Pikesville, MD (US)

(21) Appl. No.: 18/286,185

(22) PCT Filed: Apr. 8, 2022
(86) PCT No.: PCT/US22/24045

§ 371 Date: Oct. 9, 2023

Related U.S. Application Data
(60) Provisional application No. 63/173,284, filed on Apr. 9, 2021.

(51) Int. Cl. A61K 31/444; A61P 25/08; A61P 25/20

(52) U.S. Cl. CPC A61K 31/444; A61P 25/08; A61P 25/20

(57) ABSTRACT

News methods, compositions and kits that comprise perampanel are provided for treating a subject having a SYNGAP1 neurodevelopmental disorder, including to treat conditions of sleep disorder, behavior problems and/or myoclonic or reflex seizures.

The present application claims the benefit of U.S. provisional application No. 63/173,284, filed Apr. 9, 2021, incorporated herein by reference.

### BACKGROUND

SYNGAP1 gene mutations have been associated with autism or autism spectrum disorders, nonsyndromic intellectual disability, delay of psychomotor development, acquired microcephaly, and several forms of idiopathic generalized epilepsy.

SYNGAP1 is a neurodevelopmental disorder that presents with non-syndromic intellectual disability, epilepsy, sleep disorder and disruptive behaviors. See, Mignot et al. Journal of Medical Genetics. 53 (8): 511-522 (2016). A condition caused by SYNGAP1 gene mutations is called MRD5 (Mental Retardation, autosomal Dominant 5).

It would be desirable to have new therapies for conditions and disorders associated with SYNGAP1.

### SUMMARY

In a first aspect, I have now found new methods for treating a subject having a SYNGAP1 neurodevelopmental disorder.

The methods include administering an effective amount of perampanel to the subject having a SYNGAP1 neurodevelopmental disorder.

In particular aspects, the subject may be identified as suffering from or susceptible to a sleep disorder and the perampanel is administered to the identified subject.

The subject also may be identified as suffering from or susceptible to a behavioral problem and the perampanel is administered to the identified subject.

The subject also may be identified as suffering from or susceptible to a myoclonic or reflex seizure and the perampanel is administered to the identified subject.

In a preferred aspect, a compatively low dose of perampanel is administered to a subject. I have found that effective results can be achieved with such low doses.

More particularly, up to 6.0 mg of perampanel is administered to the subject per day, or up to 5.5 mg, or up to 5.0 mg, or up to 4.5 mg, or up to 4.0 mg, or up to 3.5 mg, or up to 3.0 mg, or up to 2.5 mg, or up to 2.0 mg, or up to 1.5 mg, or up to 1.0 mg, or up to 0.5 mg, or up to 0.4 mg, or up to 0.3 mg, or up to 0.2 mg, or up to 0.1 mg of perampanel is administered to a subject per day.

In another aspect, up to 6.0 mg of perampanel is administered to the subject per 2-day period (48 hours), or up to 5.5 mg, or up to 5.0 mg, or up to 4.5 mg, or up to 4.0 mg, or up to 3.5 mg, or up to 3.0 mg, or up to 2.5 mg, or up to 2.0 mg, or up to 1.5 mg, or up to 1.0 mg, or up to 0.5 mg, or up to 0.4 mg, or up to 0.3 mg, or up to 0.2 mg, or up to 0.1 mg of perampanel is administered to a subject per 2-day period (48 hours). In certain aspects, a single dose of perampanel may be administered to a subject over a 2 day (48 hour) period. In certain aspects, 2, 3 or 4 doses of perampanel may be administered to a subject over a 2 day (48 hour) period.

In another aspect, up to 6.0 mg of perampanel is administered to the subject per 3-day period (72 hours), or up to 5.5 mg, or up to 5.0 mg, or up to 4.5 mg, or up to 4.0 mg, or up to 3.5 mg, or up to 3.0 mg, or up to 2.5 mg, or up to 2.0 mg, or up to 1.5 mg, or up to 1.0 mg, or up to 0.5 mg, or up to 0.4 mg, or up to 0.3 mg, or up to 0.2 mg, or up to 0.1 mg of perampanel is administered to a subject per 3-day period (72 hours). In certain aspects, a single dose of perampanel may be administered to a subject over a 3-day (72 hours) period. In certain aspects, 2, 3 or 4 doses of perampanel may be administered to a subject over a 3-day (72 hour) period.

The perampanel may be administered on a variety of schedules. In certain protocols, a single administration per day will be suitable. In other protocols, perampanel may be administered to a subject multiple times during a 24 hour period. The administration also may be extended, for example the daily dose may be administered to a subject for several days or weeks, including 2, 3, 4, 5, 6, or 7 or more days, or 1, 2, 3, 4, 5, 6, 7 or 8 or more weeks, or 3, 4, 5, 6, 7, 8, 9, 10, 11, 12, 14, 16, 18, 20, 22 or 24 months or more.

The perampanel also may be at various times throughout a day. In certain protocols, it may be preferred to administer the perampanel to the subject in the evening (e.g. 6:00 pm, 7:00 pm, 8:00 pm or later), including to treat a sleep disorder.

In certain protocols, the subject is a pediatric patient. More particularly, in certain aspects, the subject may be 9 years old or younger. In certain aspects, the subject may be 6 years old or younger. In certain aspects, the subject may be 4 years old or younger. In certain aspects, the subject's age may be between 9 and 20 years. In certain aspects, the subject's age is between 9 and 17 years.

Pharmaceutical compositions are also provided that in general comprise a therapeutically effective of perampanel optionally together with a pharmaceutically acceptable carrier. In certain preferred aspects, the present pharmaceutical compositions contain perampanel in low dosage amount, including a pharmaceutical composition that comprises perampanel in an amount of less than 2 mg in a unit dosage oral form, or where perampanel is present in an amount of 1.5 mg, 1.0 mg, 0.9 mg, 0.8 mg, 0.7 mg, 0.6 mg, 0.5 mg, 0.4 mg, 0.3 mg, 0.2 mg or 0.1 mg in an oral dosage form.

Kits are also provided that in one aspect may comprise (a) perampanel; and (b) instructions for use of the perampanel for treatment of a condition or disorder associated with a SYNGAP1 neurodevelopmental disorder.

In another aspect, kits are provided that comprise (a) perampanel; and (b) instructions for use of the perampanel for treatment of a sleep disorder.

In a further aspect, kits are provided that comprise (a) perampanel; and (b) instructions for use of the perampanel for treatment of a behavioral problem.

In a yet further aspect, kits are provided that comprise: (a) perampanel; and (b) instructions for off-label use of the perampanel for its off label use.

In a yet further aspect, kits are provided that comprise: (a) perampanel; and (b) instructions for use of the perampanel for treatment of sleep dysfunction, motor and sensory impairment, hypotonia and behavioral disorders, and/or a myoclonic or reflex seizures.

In preferred aspects, the kits may contain perampanel in low dosage forms, such as perampanel in an amount of less than 2 mg in a unit dosage oral form, or perampanel in an amount of 1.5 mg in a unit dosage oral form, or perampanel in an amount of 1.0 mg in a unit dosage oral form, or perampanel in an amount of 0.5 mg in a unit dosage oral form, or perampanel in an amount of 0.4 mg, 0.3 mg or 0.2 mg in a unit dosage oral form.

Perampanel also may be administered to a subject, e.g. once daily, or less frequently such as once every 36, 48, 72 or 96 hours.

The kits suitably may comprise instructions for use in the form of a written package insert or package label.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All documents mentioned herein are herein incorporated by reference herein.

Other aspects of the invention are disclosed infra.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 (includes FIGS. 1A and 1B) shows data of GFP florescent staining for Orexin receptors A and B in wt vs. SynGap mutant mice reveals an increase in the number of Orexin positive neurons in the hypothalamus.

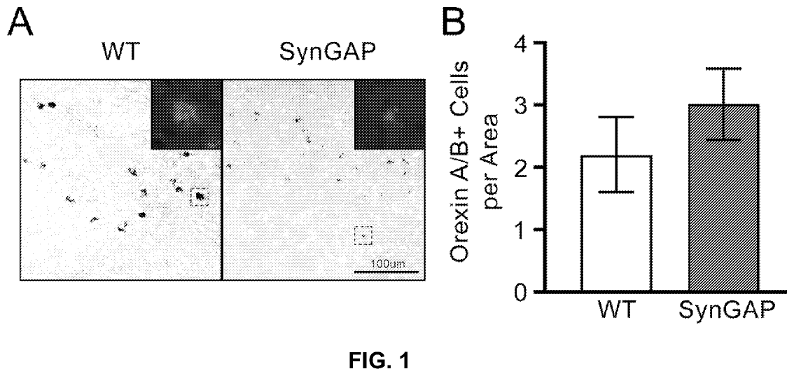

FIG. 2 shows EEG gamma power levels over a 12 hour sleep period in a 3 year old boy with SYNGAP1 haploinsufficiency.

FIG. 3 (includes FIGS. 3A-3J) shows EEG abnormalities pre- and post-PMP superimposed over hypnogram. High amplitude generalized 3-3.5 Hz spike and wave discharges (FIG. 3A) and high amplitude generalized delta activity without spikes (FIG. 3B), indicated by red and purple vertical lines on the hypnogram respectively (FIGS. C, D).The generalized discharges clustered at transition from wake to sleep and from REM to NREM sleep. Occipital rhythmic delta activity [OIRDA] (FIG. E) and focal epileptiform discharges over central and temporal head regions over the right (FIG. F), indicated by blue and green vertical lines on hypnogram respectively (FIGS. C, D). OIRDA was more prevalent in the wake period and REM sleep. It was also significantly reduced post-PMP. Focal epileptiform discharges were more prevalent post PMP compared to pre PMP. (G-J) Progression of the mean number of events per cluster when the clustering coefficient increased. Clustering coefficient determines the range of a cluster.

FIG. 4 (includes FIGS. 4A-4C) shows wake-NREM-REM sleep delta and gamma powers plotted for 16 h EEG both pre- and post-PMP over hypnograms (black line) for the same EEG show opposite trends with delta (blue line) going up in NREM slow-wave-sleep and gamma (red line) increasing in wake and REM sleep stages (FIGS. A, B). Quantification of gamma slopes for behavioral-state transitions from REM to NREM show abnormally increased gamma in the pre-PMP EEG that is significantly rescued on the post-PMP EEG in the same patient (FIG. 4C).

FIG. 5 (includes FIGS. 5A-5I) shows results of Example 8 which follows: HET^{+/-} exhibited with longer wake duration than WT^{+/+} during the exploratory phase

FIG. 6 includes FIGS. 6A-6E) shows results of Example 9 which follows: low-dose PMP rescuedgamma homeostasis in juvenile HET^{+/}.

FIG. 7 (includes FIGS. 7A-G1-2) shows results of Example 10 which follows: PMP alleviated cortical gamma dysregulation aggravated by SD.

FIG. 8 (includes FIGS. 8A-8F) shows results of Example 11 which follows: juvenile HET+/− had increased frequency of fast-active events during the exploratory phase.

FIG. 9 (includes FIGS. 9A-9E) shows results of Example 12 which follows: high nesting score, novel preference, and marble burying score in HET^{+/-} all indicated hyperactivity.

FIG. 10 (includes FIGS. 10A-10B): shows results of Example 13 which follows: EEGs in juvenile HET+/− show cortical spikes.

### DETAILED DESCRIPTION

Perampanel, (3-(2-cyanophenyl)-5-(2-pyridyl)-1-phenyl-1,2-dihydropyridin-2-one), has the following chemical structure:

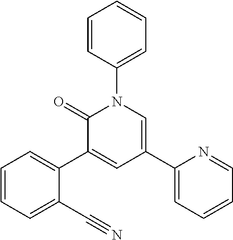 [SMILES: N#Cc1ccccc1-c1cc(-c2ccccn2)cn(-c2ccccc2)c1=O]

In certain aspects, methods are provided to treat a subject having a SYNGAP1 neurodevelopmental disorder, comprising:

identifying a subject as suffering from a SYNGAP1 neurodevelopmental disorder;

selecting the identified subject for treatment; and

administering to the identified and selected subject an effective amount of perampanel.

In certain aspects, methods are provided to treat a subject suffering from or susceptible to a sleep disorder, comprising:

identifying a subject as suffering from a sleep disorder;

selecting the identified subject for treatment; and

administering to the identified and selected subject an effective amount of perampanel. In certain aspects of such methods, the subject has a SYNGAP1 neurodevelopmental disorder.

In further aspects, methods are provided to treat a subject suffering from or susceptible to a behavioral problem, comprising:

identifying a subject as suffering from a behavioral problem;

selecting the identified subject for treatment; and

administering to the identified and selected subject an effective amount of perampanel. In certain aspects of such methods the subject has a SYNGAP1 neurodevelopmental disorder.

In still further aspects, methods are provided to treat a subject suffering or susceptible to myoclonic or reflex seizures, comprising:

identifying a subject as suffering or susceptible to myoclonic or reflex seizures;

selecting the identified subject for treatment; and

administering to the identified and selected subject an effective amount of perampanel. In certain aspects of such methods the subject has a SYNGAP1 neurodevelopmental disorder.

The subject to be administered with perampanel as disclosed herein is suitably a mammal, particularly a human, including human having an age (U.S. convention for age) of 20, 19, 18, 17, 16, 15, 14, 13, 21, 11, 10, 9, 8, 7, 6, 5, 4, 3 or 2 years or less.

In some embodiments, the therapeutically effective dose of perampanel may be administered in combination with one or more other distinct therapeutics. For example, an orexin receptor antagonist such almorexant, lemborexant, SB-334,867, SB-408,124, SB-649,868, and/or suvorexant may be administered in combination or conjunction with perampanel. In certain embodiments, lemborexant may be administered in combination or conjunction with perampanel. In certain aspects, an orexin A/B receptor antagonist (e.g. lemborexant) may be administered in combination or conjunction with perampanel.

In one preferred aspect, a present formulation of the invention comprising perampanel may be used in combination with or include one or more other therapeutic agents or dietary or nutritional supplements and may be administered either sequentially or simultaneously by any convenient route in separate or combined pharmaceutical compositions. As used herein, combination of two or more compounds may refer to a composition wherein the individual compounds are physically mixed or wherein the individual compounds are physically separated. A combination use encompasses administering the components separately to produce the desired additive, complementary or synergistic effects. In certain exemplary embodiments, perampanel and one or more additional therapeutic agents are physically mixed in the composition. In additional exemplary embodiments, perampanel and one or more additional therapeutic agents are physically separated in the composition.

The therapeutically effective dose of perampanel can be administered to the subject by a variety of administration routes. Oral administration will be typically preferred although other administration protocols also may be utilized. In some embodiments, the compound may be formulated for administering purposes in a capsule, a tablet, a gel, a powder, liquid, suspension or emulsion.

As discussed, therapeutic compositions are also provided that include perampanel optionally with a pharmaceutically acceptable carrier.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

In one preferred aspect, perampanel may be formulated for administering purposes in a capsule, a tablet, a gel, a powder, liquid, suspension or emulsion; however, the administering methods may not be particularly limited.

As discussed, perampanel can be included in a kit, container, pack, or dispenser together with instructions for administration. For instance, the kit may contain a product label or written package insert that discloses use of the composition for treating including prophylaxis of a disorder as disclosed herein.

The following non-limiting examples are illustrative.

### EXAMPLE 1

A primary role of orexins is to control sleep and arousal, and the neurons that release orexins are most active during the day. To keep us awake, these neuropeptides stimulate other neurons to release neurotransmitters that promote alertness during wake states. The current hypothesis is that in epileptic brains the expression of orexin goes up resulting in sleep dysfunction associated with inability to fall asleep and frequent awakenings.

Both these sleep problems are heavily reported in SYNGAP1 patients who are empirically given sedative drugs to help their sleep which often fails to improve the sleep problems but additionally causes drowsiness.

The administration of perampanel to a subject as disclosed herein, including in combination with lemborexant (orexin A/B receptor antagonist) can improve sleep in subjects without causing drowsiness which can be important or critical to allow day-time learning in young children.

Preliminary data show increase in the number of orexin positive neurons in the mutant mice and we have previously reported the loss of light and dark cycle sleep modulation in the mutants in 24 h EEGs.

To correct cortical gamma oscillations, perampanel can be administered to a subject as disclosed herein to prevent or reduce night time awakenings, preferably together with co-administration of lemborexant.

### EXAMPLE 2

We have shown that low dose Perampanel (PMP) directly targets PV+ interneurons and was rescue the excessive AMPA insertion and hyperexcitable phenotype in SynGAP1 mice.

### EXAMPLE 3: HIGH GAMMA DURING SLEEP IN A SYNGAP1 PATIENT

Sleep EEG analysis of EEG gamma power during overnight EEG reveals significantly high gamma power during NREM when typically gamma power is lower compared to REM.

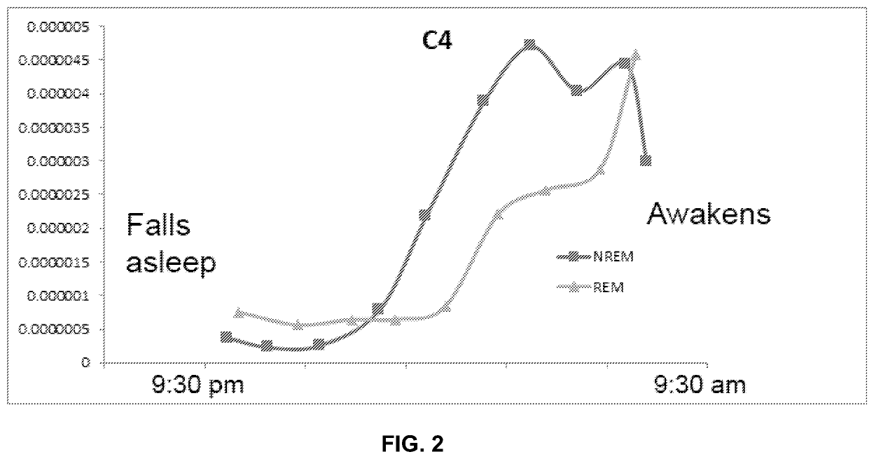

Results are summarized in FIG. 2 which shows EEG gamma power levels over a 12 hour sleep period in a 3 year old boy with SYNGAP1 haploinsufficiency. Leads represented are the C4 lead in a sleep EEG recorded with the 10-20 standard recording system. The child did not wake up during this period as documented on video and has normal NREM cycles for the slow-wave sleep cycles associated with delta power. The gamma power oscillations that are higher in first few hours of sleep reverse such that gamma is higher in NREM and lower during REM in this SYNGAP1 EEG. The reversal was detected in the second half of the sleep period that started at 9:30 pm and ends at 9:30 am.

These findings validate the qEEG biomarker identified in the HET syngap1 mouse. See Sullivan et al., Biol Psychiatry 2020 May 1;87(9):829-842.

### EXAMPLE 4

A clinical protocol of perampanel in SYNGAP1 patients aged 2-17 years over 28 days includes procedures and analysis according to the following schema:

### EXAMPLE 5: TREATMENT PROTOCOL

A 2 year old human child diagnosed with SYNGAP1 and experiencing persistent poor sleeping is administered 2 mg tablet of Perampanel shortly before bedtime once a day over 16 weeks.

### EXAMPLE 6: ADDITIONAL TREATMENT PROTOCOL

A 2 year old human child diagnosed with SYNGAP1 and experiencing persistent poor sleeping is administered oral syrup with 0.2 mg of Perampanel shortly before bedtime once a day slowly ramped up to 1.5 mg per day (3 ml syrup) over 12 months.

### EXAMPLE 7: CASE STUDY

A 25-month-old girl daughter with pathogenic SYNGAP1 variant was trialed on low doses of PMP.

PMP is currently approved for focal onset seizures with or without evolution to bilateral tonic clonic seizures in children ≥4 years of age and as an adjunctive treatment of primary generalized tonic clonic seizures in children ≥12 years of age. The recommended daily maintenance dose range is 8-12 mg daily.

The child has now been on low-dose PMP (<2 mg daily) for over one year. We here discuss the safety profile, changes in the electroencephalographic (EEG) features and clinical features, especially in the developmental profile and sleep pre-and-post PMP. We hypothesize that PMP at doses lower than typically used as an antiseizure medication will be safe in patients with SYNGAP1-DEE and help normalize sleep and behavioral disorders. Almost 50% of SYNGAP1-DEE related epilepsies are refractory to treatment from the very onset and currently very few children are prescribed PMP even for seizure control such that only 1 of 57 patients in a cohort study was documented to have been on it(3).

### Methods

Clinical data from physician's clinic notes; genetic testing reports; developmental scores from occupational therapy, physical therapy, speech and language therapy evaluations; and applied behavioral analysis (ABA) reports were reviewed.

### Electroencephalogram (EEG) Analysis

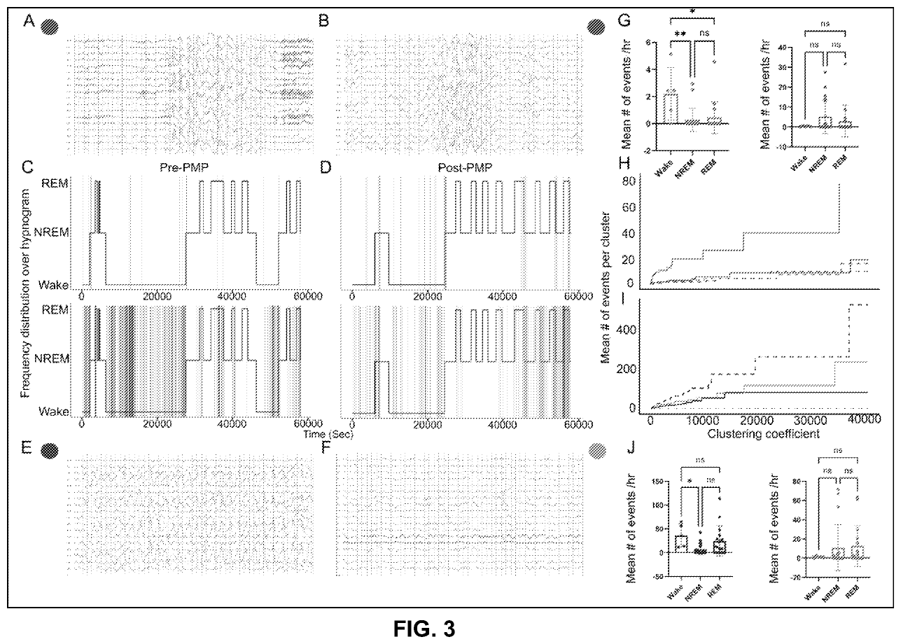

EEGs used for analysis were recorded with a 10-20 electrode placement system. EEGs were reviewed in Persyst (version 14, Persyst Development Corporation, Prescott, AZ). Long-duration continuous EEGs (≥16 h) that included awake and sleep states were reviewed in non-overlapping 10-second epochs. Each epoch was analyzed for the presence or absence of abnormalities and were given a binary score (1=present, 0=absent). The findings that were scored were abnormal slowing (generalized or focal, FIGS. 3A, B) and epileptiform discharges (generalized or focal, FIGS. 3C,D).

### Cluster Analysis of Abnormal Events

In order to characterize and quantify the clustered nature of spikes, hierarchical clustering analysis was implemented. Hierarchical clustering analysis groups data points based on Euclidean distance and the size of clusters were determined by the clustering coefficient. Average number of spike events in a cluster was computed for a sequence of increasing clustering coefficients, and the progressions for spike subtypes were compared. The rate at which the average spike events increased with respect to clustering coefficient and the size characterized the clustered nature of spike events.

### Hypnogram and Quantitative EEG Analysis

Sleep stages were manually scored in 10 second epochs. FFT was applied to 10 second epochs in Sirenia Sleep software (Pinnacle Tech. Inc, Kansas USA) for further EEG analysis. Previous reports suggest that SYNGAP1 mice displayed abnormal gamma trends during behavioral transitions between wake and sleep (Sullivan et al., 2020). To analyze the presence of similar gamma dysregulation in REM and NREM transition points, linear regression analysis was done on 10 min segment of the REM to NREM transition point

### Case Description

Patient was born at 37 weeks gestation to non-consanguineous parents of Chinese descent, mother aged 34 years and father aged 38 years. She was born by normal spontaneous vaginal delivery, after an uncomplicated pregnancy. Her birth weight was 3.19 kg and head circumference was 33 cm. There were no complications immediately after delivery except for mild hyperbilirubinemia, not requiring phototherapy. The patient was generally healthy, but by seven months of age, developmental delays across several domains became apparent. On physical exam, there were no dysmorphic features identified; however, marked axial and appendicular hypotonia with diffusely diminished deep tendon reflexes were noted. At seven months of age she received physical therapy, occupational therapy, and developmental therapy through early intervention program. During formal developmental evaluation at 24 months of age using the Hawaii Early Learning Profile (H.E.L.P.) instrument she was found to be delayed in her cognitive skills (25% delay), both receptive and expressive communication skills (50% and 58% delay respectively), gross motor skills (58% delay), fine motor skills (25% delay), social and emotional skills (50% delay) and also adaptive skills (50% delay). During motor assessment particularly, the patient was not able to stand or walk independently. During formal gross and fine motor assessment using Peabody Developmental Motor Scales, 2^{nd} Edition (PDMS2) she showed significant delays in the following subsets—stationary (42% delay), locomotion (50% delay) and object manipulation ((50% delay). Lastly, she exhibited signs and symptoms of autism and was eventually diagnosed with autism. She underwent genetic testing in order to find an underlying etiology for the global developmental delay and autism. Her whole exome sequencing revealed a pathogenic variant in the SYNGAP1 gene (c.1167delA,p.G391AfsX12, heterozygous).

The diagnosis of SYNGAP1-DEE prompted a routine electroencephalogram (EEG) at 18 months of age since the majority of patients with SYNGAP1-DEE have epilepsy⁴. The interictal EEG showed high voltage generalized-spike-wave discharges with an occipital predominance. An overnight continuous 16-hour EEG done at 19 months of age showed occasional bursts of high voltage 2.5-3 Hz generalized spike-and-and-slow-wave activity with a occipital predominance (O1,O2>P7,P8) particularly in the transition zones between awake to sleep; additionally, seen during awake to sleep transitions were high voltage semirhythmic to rhythmic 2-3 Hz delta intrusions lasting about two seconds which gradually increased in prevalence giving way to near continuous high amplitude semirthythmic delta activity in sleep; finally, there were frequent runs of medium to high voltage 2.5-3.5 Hz notched rhythmic delta activity without evolution or clinical correlation, consistent with occipital intermittent rhythmic delta activity (OIRDA) which at times exhibited fixation-off-phenomenon. Occasionally, the rhythmic delta activity had a more generalized appearance with an occipital predominance. These runs of occipital rhythmic delta activity were most prevalent during the awake state and during REM sleep. Lastly, rare focal epileptiform discharges over the temporal and central head regions were seen bilaterally. The parents first noted clinical seizures when she was 22 months of age which were described as episodes of eye blinking (eyelid myoclonia) that occurred daily. The patient subsequently also developed atonic seizures with “head-drops” at around 38 months of age. Polysomnogram at 22 months of age revealed mild obstructive sleep apnea.

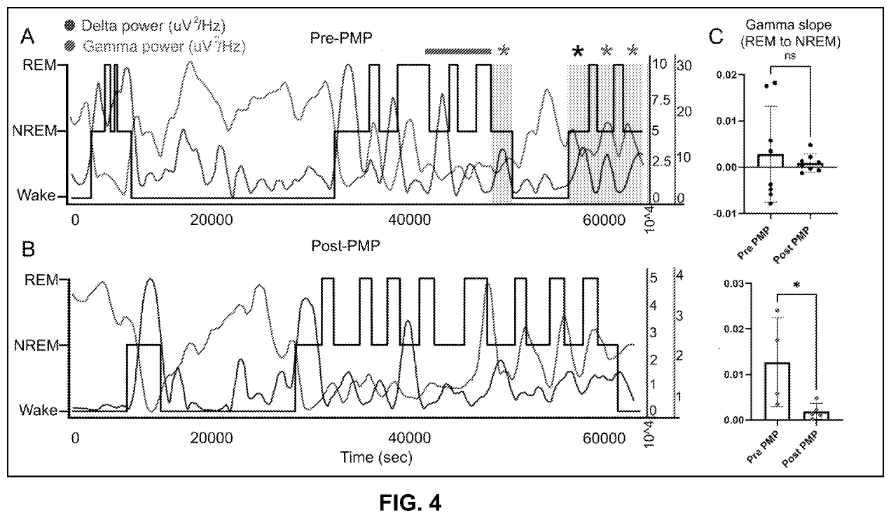

Low-dose PMP at 0.2 mg every night was started when she was 25 months of age. The dose was subsequently increased to 0.3 mg every night. The patient tolerated the PMP well without any side effects. At 6 months post-initiation of treatment with once daily low-dose PMP a bedtime, she showed significant improvements in her tone and strength, as noted during her physical therapy evaluation. Additionally, she started walking independently, climbing a variety of surfaces and exploring her environment. Occupational therapy evaluation revealed an improvement in her fine and visual motor skills. Parent also reports improvement in sleep quality with fewer night time awakenings. The Verbal Behavior Milestones Assessment and Placement Program (VB-MAPP) which is an assessment and skills-tracking system to assess the language, learning and social skills of children with autism or other developmental disabilities was used to evaluate her language and other related social skills. An overall improvement from baseline was noted in cognition, communication, social and behavioral domains as summarized in Table1. Patients' baseline VB-MAPP score at 21 months of age (prior to starting PMP) was 7/45, which improved to 10/45 at 3 months post PMP and then to 17.5/45 at 10 and 13 months post PMP with 37.2% of developmental goals mastered. A follow-up overnight EEG was done at 29 months of age. Several differences were noted in her EEG. Firstly, an overall decrease in the prevalence of the OIRDA in wake and REM sleep was observed. Multifocal epileptiform discharges, which were rarely observed in her prior EEGs, were noted over the temporal and central head regions bilaterally and occurred independently. At times the centro-temporal discharges occurred synchronously. These discharges were most prevalent in the N2 sleep, which increased in prevalence in subsequent N2 sleep cycles (FIG. 3). Finally, similar to dysregulated gamma seen during behavioral transition points from wake to NREM sleep in Syngap1^{+/-} mice, we also observed gamma dysregulation in our patient's pre-PMP EEG, where gamma power was increased during NREM when transitioning from REM and, therefore, presenting a positive slope (FIG. 4). This abnormality was significantly alleviated after the PMP (FIG. 4), similar to what was observed in the preclinical studies.

### Discussion

A young patient underwent the clinical study who exhibited a novel pathogenic variant in SYNGAP1 who exhibited global developmental delays by 7 months of age and developed clinical seizures at around 22 months of age involving eyelid myoclonic seizures, and later atonic seizures with head drops. Of note, epileptiform discharges were noted in the EEG prior to the onset of clinical seizures. Based on the preclinical evidence of low-dose PMP significantly rescuing impaired behavioral-state cortical gamma homeostasis in Syngap1+/− mice, she was trialed with low-dose PMP. After starting PMP at low doses (0.2 mg−>0.3 mg per day at bedtime), the patient showed significant improvements in motor (gross motor, fine motor and visual motor), cognition, communication, social and behavior domains. Low dose PMP was not helpful in controlling her clinical seizures, similar to the findings reported in the mouse model. Low-dose PMP did not have any significant effect on the epileptiform discharges; moreover, the EEG evolved from initially with just demonstrating generalized epileptiform discharges and rare focal epileptiform discharges to consisting generalized and frequent multifocal epileptiform discharges in follow-up EEG.

The seizure types in our patient included eyelid myoclonias and atonic head drops which have been described in prior SYNGAP1 series. The most common type of seizures in SYNGAP1-DEE are generalized seizures including myoclonic, atonic, and myoclonic-atonic seizures; atypical absences; eyelid myoclonia and myoclonic absences(1,3,9-11) . One patient in the series of fifty-seven patients reported by Vlaskamp DRM et al. was described with West syndrome. Photosensitivity, eye-closure sensitivity and fixation-off sensitivity (FOS) is reported in some individuals with SYNGAP1-DEE. Other seizure triggers including eating, sounds and touch have also been described(3,10-13)

The EEG features in the patient including generalized spike-and-slow-wave discharges with an occipital predominance, multi-focal epileptiform discharges, generalized rhythmic delta activity and OIRDA with or without FOS are also shared by patients from various SYNGAP1 series. Overall, generalized epileptiform discharges are more common than focal epileptiform discharges. The generalized epileptiform discharges tend to have an occipital predominance(12,14). Focal epileptiform discharges are typically multifocal, but again an occipital predominance has been reported in several patients in different reports(3,10-15). Upon plotting the EEG abnormalities and epileptiform activity over a graph of behavioral state changes, we found that the generalized epileptiform discharges and generalized rhythmic delta activity clustered at sleep onset. This phenomenon was also described in prior reports(14). Intermittent rhythmic delta activity is another common finding in patients with a pathogenic SYNGAP1 variant(12,14). The delta activity often seen in the posterior head regions or are generalized with an occipital/posterior predominance. Specifically, OIRDA which is often notched in morphology has been described in prior reports of patients with SYNGAP1 pathogenic variant. In a series of 15 patients described by Lo Barco et al. 14/15 patients had a notched OIRDA that exhibited fixation off sensitivity and became near continuous while falling asleep and during initial phases of sleep(12). Similar features were also seen in our patient.

In neurodevelopmental disorders, development of precise disease modifying agents requires simultaneous identification of objective and sensitive biomarkers to help stratify the genetically and phenotypically heterogeneous patients so as to enrich population for maximal treatment response. Furthermore, biomarkers that reflect molecular target engagement and measures treatment response may serve as early indicators of treatment efficacy compared to traditional endpoints. Neurophysiologic biomarkers using studies such as EEG which is a non-invasive and readily available tool holds significant promise especially given its high temporal resolution (16,17). Jimenez-Gomez et al. aimed to identify neurophysiologic biomarkers in patients with pathogenic SYNGAP1 variants(14). They found a moderate correlation between developmental age equivalence in language and visual-perceptual/fine motor (VP/FM) development with frequency of posterior basic rhythm. Additionally, they reported that the presence of intermittent rhythmic delta activity did not correlate with the severity of developmental disability. To date PMP has rarely been prescribed in SYNGAP1 related seizures (1 out of 57 patient cohorts)(3). The general impression for its use as an ASM has been the occurrence of side-effects as the dose is increased every 2-3 weeks to its standard dosing of >4-6 mg which usually results in discontinuation. Cortical gamma homeostasis plays a significant role in sleep quality, cognition and learning²¹. The rational for using low-dose PMP here is 1st off-label use to help reverse sleep, cognitive and behavioral symptoms in SYNGAP1-DEE. Periods of wakefulness marked by high cognitive load is associated with high gamma power, which typically decreases when transitioning from awake to sleep. PMP is a pan AMPAR blocker(18). PMP-treated Syngap1+/− mice showed a significant rescue of the gamma dysregulation. On the other hand, PMP had a weaker effect on interictal spikes suppression in the epileptic mice with the low dose(5). Similarly, our patient demonstrated a rescue of cortical gamma dysregulation and marked improvement in developmental domains after treatment with PMP; however, it had no effect on interictal epileptiform discharges or clinical seizures.

In conclusion, this is the first case report of a patient who was safely and successfully treated with low-dose PMP, without significant side-effects and has shown improvement in developmental measures and sleep. Her EEG has shown significant rescue of a novel qEEG biomarker identified through preclinical research; gamma homeostasis at sleep transition points, on low-dose PMP. She is currently on a dose of 1.5 mg daily at bed-time and has been tolerating it well. Additionally, she was also started on intensive therapies at 7 months of age, which may also contribute to the overall trajectory of improvement. The strong preclinical data on rescue of cortical gamma dysregulation with use of low-dose PMP in conjunction with this case report highlights the off-label use of low-dose PMP to target the sleep, cognitive and behavioral symptoms seen in SYNGAP1-DEE and the treatment of the associated epilepsy would require an add on ASM at its recommended doses for efficacy.

Results are also summarized in the following Table 1:

TABLE 1
VB-MAPP assessment for verbal and social skills
(↑ = improvement in skill from prior assessment,
↓ = worsening in skill from prior assessment,
NA = not assessed).

3 months post → 10 months post

| | PMP | PMP |
|---|---|---|
| VB-MAPP Domain | (dose 0.2 mg) | (dose 0.3 mg) |
| Communication | | |
| Manding | ↑ | ↓ |
| Pointing/gesturing | ↑ | ↑ |
| to preferred items | | |
| Listener | ↑ | ↓ |
| Responding- | | |
| reinforcing/ | | |
| preferred items | | |
| PECS-Phase 1 | ↑ | ↑ |
| Cognitive | | |
| Self-help | NA | NA |
| Motor imitation | ↑ | ↑ |
| Receptive | ↑ | ↑ |
| commands | | |
| Social | | |
| Responds to name | ↑ | ↑ |
| Parallel play | ↑ | ↑ |
| Independent play | ↑ | ↑ |
| Health | | |
| Feeding | ↑ | ↑ |
| Behavior | | |
| Functional | ↑ | ↑ |
| Communication | | |
| training | | |
| Waiting | ↑ | ↑ |

### REFERENCES OF EXAMPLE 7

1. Hamdan F F, Gauthier J, Spiegelman D, Noreau A, Yang Y, Pellerin S, et al. Mutations in SYNGAP1 in autosomal nonsyndromic mental retardation. New England Journal of Medicine. 2009;360(6):599-605.
2. Hamdan F F, Daoud H, Piton A, Gauthier J, Dobrzeniecka S, Krebs M-O, et al. De novo SYNGAP1 mutations in nonsyndromic intellectual disability and autism. Biol Psychiatry. 2011 May 1;69(9):898-901.
3. Vlaskamp D R M, Shaw B J, Burgess R, Mei D, Montomoli M, Xie H, et al. SYNGAP1 encephalopathy: A distinctive generalized developmental and epileptic encephalopathy. Neurology. 2019 Jan 8;92(2):e96-107.
4. Kim J H, Lee H-K, Takamiya K, Huganir R L. The role of synaptic GTPase-activating protein in neuronal development and synaptic plasticity. J Neurosci. 2003 Feb 15;23(4):1119-24.
5. Sullivan B J. Low-dose Perampanel rescues cortical gamma dysregulation associated with parvalbumin interneuron GluA2 upregulation in epileptic Syngap1+/− mice—ScienceDirect [Internet]. [cited 2020 Jan 21]. Available from: https://www.sciencedirect.com/science/article/pii/S0006322320300020
6. Sohal V S, Zhang F, Yizhar O, Deisseroth K. Parvalbumin neurons and gamma rhythms enhance cortical circuit performance. Nature. 2009 Jun;459(7247):698-702.
7. Vyazovskiy V V, Cirelli C, Pfister-Genskow M, Faraguna U, Tononi G. Molecular and electrophysiological evidence for net synaptic potentiation in wake and depression in sleep. Nat Neurosci. 2008 Feb;11(2):200-8.
8. Zhu J J, Qin Y, Zhao M, Van Aelst L, Malinow R. Ras and Rap control AMPA receptor trafficking during synaptic plasticity. Cell. 2002 Aug 23;110(4):443-55.
9. Zollino M, Gurrieri F, Orteschi D, Marangi G, Leuzzi V, Neri G. Integrated analysis of clinical signs and literature data for the diagnosis and therapy of a previously undescribed 6p21.3 deletion syndrome. Eur J Hum Genet. 2011 Feb;19(2):239-42.
10. Berryer M H, Hamdan F F, Klitten L L, Møller R S, Carmant L, Schwartzentruber J, et al. Mutations in SYNGAP1 Cause Intellectual Disability, Autism, and a Specific Form of Epilepsy by Inducing Haploinsufficiency. Human Mutation. 34(2):385-94.
11. von Stülpnagel C, Funke C, Haberl C, Hörtnagel K, Jüngling J, Weber Y G, et al. SYNGAP1 Mutation in Focal and Generalized Epilepsy: A Literature Overview and A Case Report with Special Aspects of the EEG. Neuropediatrics. 2015 Aug;46(4):287-91.
12. Lo Barco T, Kaminska A, Solazzi R, Cancés C, Barcia G, Chemaly N, et al. Syngap1-Dee: a visual sensitive epilepsy. Clinical Neurophysiology [Internet]. 2021 Feb 3 [cited 2021 Feb 10]; Available from: https://www.sciencedirect.com/science/article/pii/S1388245721000353
13. Mignot C, Stülpnagel C von, Nava C, Ville D, Sanlaville D, Lesca G, et al. Genetic and neurodevelopmental spectrum of SYNGAP1-associated intellectual disability and epilepsy. Journal of Medical Genetics. 2016 Aug 1;53(8):511-22.
14. Jimenez-Gomez A, Niu S, Andujar-Perez F, McQuade E A, Balasa A, Huss D, et al. Phenotypic characterization of individuals with SYNGAP1 pathogenic variants reveals a potential correlation between posterior dominant rhythm and developmental progression. J Neurodev Disord. 2019 Aug 8;11(1):18.
15. Carvill G L, Heavin S B, Yendle S C, McMahon J M, O'Roak B J, Cook J, et al. Targeted resequencing in epileptic encephalopathies identifies de novo mutations in CHD2 and SYNGAP1. Nature Genetics. 2013 Jul;45(7):825-30.
16. Ewen J B, Sweeney J A, Potter W Z. Conceptual, Regulatory and Strategic Imperatives in the Early Days of EEG-Based Biomarker Validation for Neurodevelopmental Disabilities. Front Integr Neurosci [Internet]. 2019 [cited 2021 Aug 26];0. Available from: https://www.frontiersin.org/articles/10.3389/fnint.2019.00045/full
17. Sahin M, Sweeney J A, Jones S R. Editorial: Biomarkers to Enable Therapeutics Development in Neurodevelopmental Disorders. Front Integr Neurosci. 2020;14:616641.
18. Hanada T. The discovery and development of perampanel for the treatment of epilepsy. Expert Opinion on Drug Discovery. 2014 Apr;9(4):449-58.

### EXAMPLE 8: SLEEP BOUT ANALYSIS: JUVENILE HET+/− MICE HAVE ALTERED SLEEP ARCHITECTURE

### Methods for Examples 8-13

24 h tethered vEEG recording was performed for Het+/− mice and their age- and sex matched WT littermates at P21-P30, followed by telemetric vEEG for 24 h with 6 h SD at P35. Quantitative EEG (qEEG) analysis included the frequency bands: delta (0.5-4.0 Hz), theta(5.5-8.0 Hz), alpha (8.0-13.0 Hz), beta (13.0-30 Hz), and gamma (35-50 Hz). Linear regression of gamma frequency power during transition states from wake to sleep was quantified. Spike frequency over 24 h EEG was scored by a blind reviewer based on previously published parameters. The effect of low-dose PMP (2 mg/kg, IP, BD), an AMPA receptor antagonist, on these potential qEEG biomarkers was investigated. Additionally, movement analysis was performed using infrared cameras to trace fast-active movements between 5 am-7 am. 24 h telemetric vEEG was recorded during marble burying and novel object behavioral tests.

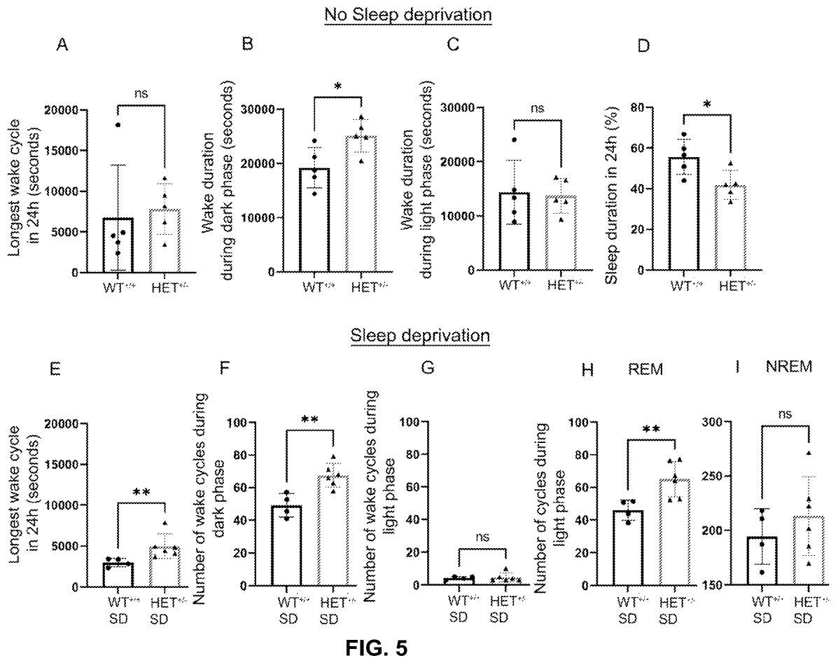

As shown in FIG. 5: HET^{+/-} exhibited with longer wake duration than WT^{+/+} during the exploratoryphase. FIG. 5A: Prior to SD, the longest wake cycle is constant between WT^{+/+} and HET^{+/-}. FIG. 5B: HET^{+/-} had longer duration of wake during the dark phase (WT^{+/+} vs. HET^{+/-}: unpaired t-test; p<0.01) (FIG. 5C) while there was no difference during the light phase. FIG. 5D: HET+/− had shorter durationof sleep than WT^{+/+} during a 24 h recording (WT^{+/+} vs. HET^{+/-}: unpaired t-test; p<0.05). FIG. 5E: Unlike prior to 6 h SD, HET^{+/-}SD had significantly increased longest wake cycle than WT^{+/+}(WT^{+/+}SD vs. HET^{+/-}SD: unpaired t-test; p<0.01). FIG. 5F: Moreover, the number of wake cyclesduring the dark phase was significantly increased in HET^{+/-}SD (WT^{+/+}SD vs. HET^{+/-}SD:unpaired t-test; p<0.01), while (FIG. 5G) it was constant during the light phase (WT^{+/+}SD vs. HET^{+/-}SD: unpaired t-test). Overall, HET^{+/-} had less sleep during the dark phase than WT^{+/+}. FIGS. 5H-I: Additionally, HET^{+/-}SD had increased frequency of REM cycles during the light phase (WT^{+/+}SD vs. HET^{+/-}SD: unpaired t-test; p<0.01), while the frequency of NREM cycles was constant.

### EXAMPLE 9: CORTICAL GAMMA DYSREGULATION: CORTICAL GAMMA DYSREGULATION APPEARS IN JUVENILE HET^{+/-}

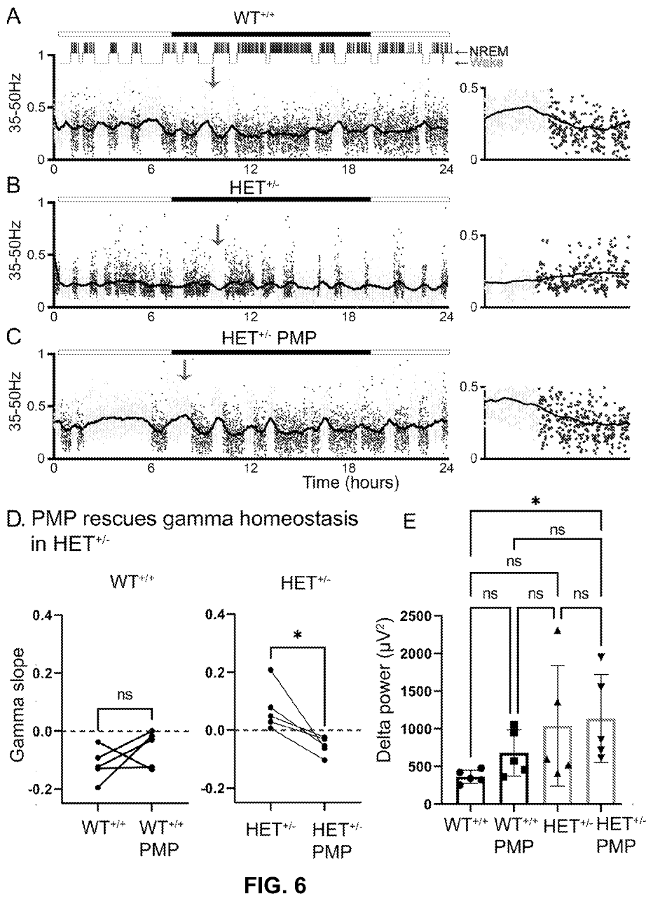

As shown in FIG. 6, low-dose PMP rescuedgamma homeostasis in juvenile HET^{+/-}. FIG. 6A: Representative 24 h hypnogram and WT^{+/+} 10 second epoch of a 24 h baseline vEEG with a loess trendline in black. The 10 s epoch data was max-min normalized in the figure. Yellow and blue denote wake and sleep-state, respectively. The light/dark phase was noted on top with a white/black bar. On the right is a representative homeostatic gamma slope that decreased from wake to sleep at a transition point noted with a red arrow. FIG. 6B: Representative gamma dysregulation in HET^{+/-} where the gamma power did not decrease from wake to NREM sleep. FIG. 6C: Representation of low-dose PMP (2 mg/kg, IP, BD) rescuing gamma homeostasis in HET^{+/-}. FIG. 6D: PMP significantly reduced the gamma slope and rescued the gamma homeostasis in behavioral transition points (HET^{+/+} vs. HET PMP^{+/-}: paired t-test; p<0.05). FIG. 6E: Delta power during NREM did not show notable statistical significance across all groups (One-way ANOVA).

### EXAMPLE 10: SLEEP DEPRIVATION (SD): SD AGGRAVATES CORTICAL GAMMA DYSREGULATION IN JUVENILE HET^{+/-}

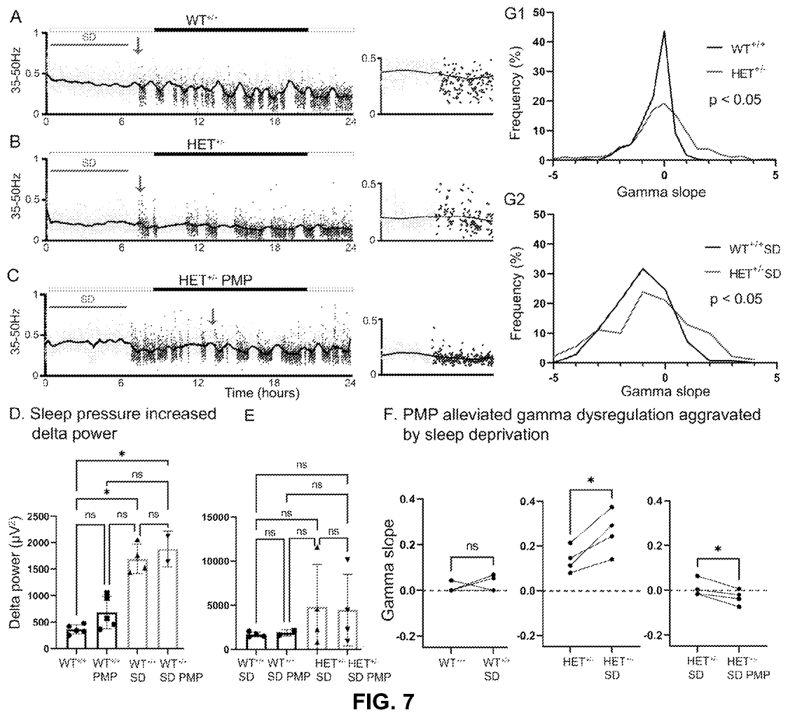

As shown in FIG. 7, PMP alleviated cortical gamma dysregulation aggravated by SD. FIG. 7A: WT^{+/+} 10 second epoch of a 24 h vEEG with 6 h SD. SD segment is marked in red. FIG. 7B: HET^{+/-} displayed increased gamma slope from wake to sleep after SD. FIG. 7C: low-dose PMP (2 mg/kg, IP) rescued gamma dysregulation during sleep transition points in HET^{+/-} after 6 h SD. FIG. 7D: The effect of 6 h SD was confirmed by comparing the delta frequency power before and after SD in WT^{+/+}. As expected, the delta frequency power increased with 6 h SD(WT^{+/+} vs. WT^{+/+}SD: One-way ANOVA; p<0.05). FIG. 7E: Delta power during NREM did not show notable statistical significance across all groups (One-way ANOVA). FIG. 7F: Cortical gamma dysregulation was aggravated by 6 h SD as the magnitude of abnormal positive gamma slope further increased (HET^{+/-} vs. HET^{+/-}SD: paired t-test; p<0.05). PMP rescued gamma homeostasis by decreasing the most dominant slope in sleep deprived HET^{+/-} mice (HET^{+/-}SD vs. HET^{+/-}SD PMP^{+/-}: paired t-test; p<0.05). FIG. 7G1-2: Histogram of gamma slope from REM to NREM before and after SD in WT^{+/+} and HET^{+/-}. Following a similarpattern, HET^{+/-} had broader positive tail in gamma slope in with and without SD (WT^{+/+} vs. HET^{+/-}: one-wayANOVA; p<0.05. WT^{+/+}SD vs. HET+/−SD: one-way ANOVA; p<0.05).

### EXAMPLE 11: BEHAVIORAL ANALYSIS: JUVENILE HET+/− MICE DISPLAYS HYPERACTIVITY IN MOVEMENT ANALYSIS

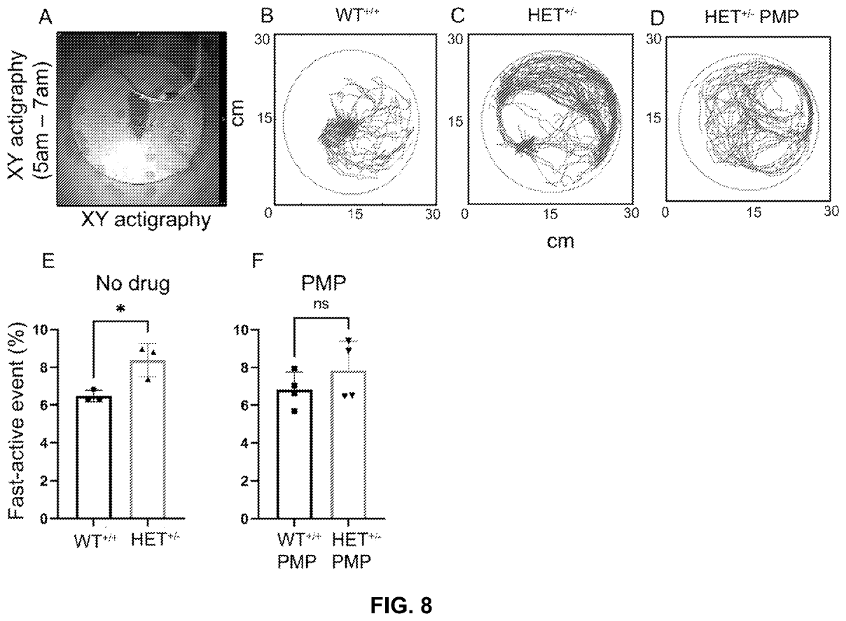

As shown in FIG. 8: juvenile HET+/− had increased frequency of fast-active events during the exploratory phase. FIG. 8A: Representative image of infrared camera used for movement analysis during the dark cycle. Hyperactivity was measured by tracing the fast-active events. FIGS B-D: 2 h representative traces of WT^{+/+}, HET^{+/-}, and HET^{+/-} with PMP activity plots, respectively (5-7 am). (E-F)HET^{+/-} had significantly increased level of fast active events compared to WT^{+/+}, and this relation was lost after the low-dose PMP administration (WT^{+/+} vs. HET^{+/-}: unpaired t-test; p<0.05).

### EXAMPLE 12: NESTING BEHAVIOR, MARBLE BURYING, AND NOVEL OBJECT TESTS CONFIRM HYPERACTIVITY

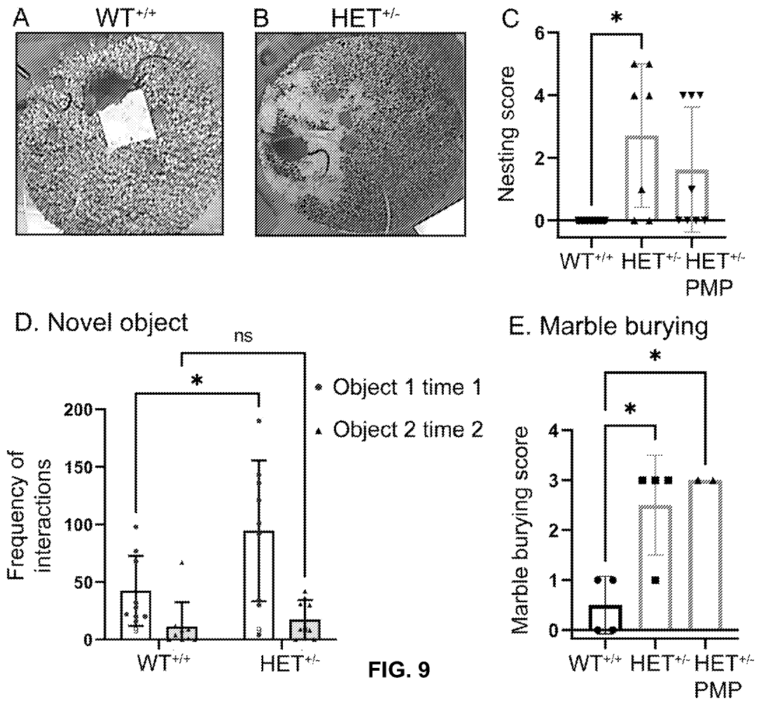

Results are shown in FIG. 9: high nesting score, novel preference, and marble burying score in HET^{+/-} all indicated hyperactivity. FIG. 9A: Representative image of a nest of WT^{+/+} (FIG. 9B) and HET^{+/-} after 24 h recording. FIG. 9C: Score was given based on the surface area of the nest. HET^{+/-} had significantly higher score compared to WT^{+/+} (WT^{+/+} vs. HET^{+/-}: unpaired t-test; p<0.05). FIG. 9D: In a novel object test, HET^{+/-} had more interactions with the initial novel object than WT^{+/+} (WT^{+/+} vs. HET^{+/-} at object 1 time 1: two-way ANOVA; p<0.05). FIG. 9E: Additionally, marble burying testing showed hyperactivity in HET^{+/-} (WT^{+/+} vs. HET^{+/-}: unpaired t-test; p<0.05).

### EXAMPLE 13: CORTICAL SPIKES ON EEG: JUVENILE HET+/− MICE EXHIBIT EEG CORTICAL SPIKES

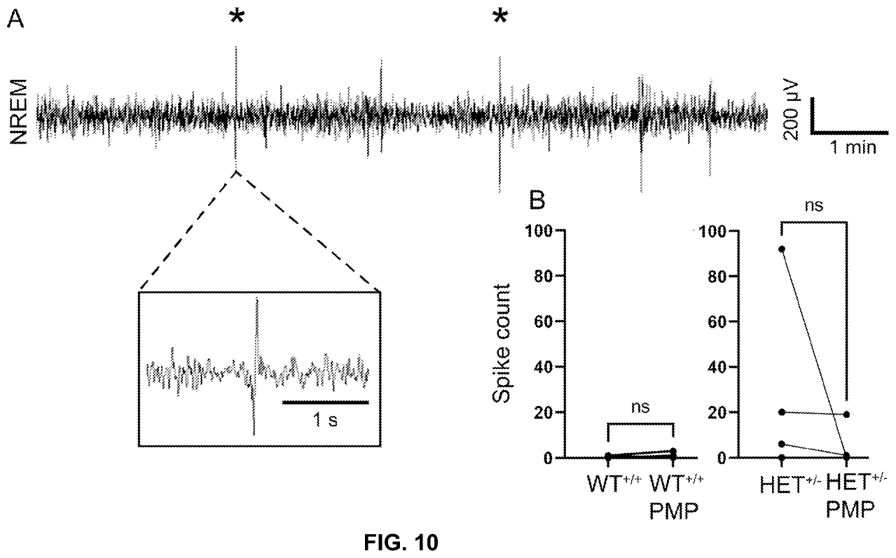

Results are shown in FIG. 10: EEGs in juvenile HET+/− show cortical spikes. FIG. 10A: Representative EEG trace of a P24 HET^{+/-} with cortical spikes (denoted by *) detected during NREM. (B) Low-dose PMP reduced spike frequency in high spiking HET^{+/-} mice however group data was not statistically significant due to variability of low-spiking HET+/− s at juvenile ages.

The above examples show:
Sleep bout analysis of 24 h EEGs showed altered sleep architecture in juvenile Syngap1^{+/-} mice.
Cortical gamma dysregulation was present in the juvenile Syngap1^{+/-} mice.
PMP also alleviated cortical gamma dysregulation aggravated by SD.
Juvenile Syngap1^{+/-} mice showed significant hyperactivity.
Cortical spikes were evident in EEGs of juvenile Syngap1^{+/-} mice

### REFERENCES EXAMPLES 8-13

1. Sullivan B J, Ammanuel S, Kipnis P A, Araki Y, Huganir R L, Kadam S D. Low-dose 381 Perampanel rescues cortical gamma dysregulation associated with parvalbumin 382 interneuron GluA2 upregulation in epileptic Syngap1^{+/-} mice. Biological Psychiatry. 2020; 383 87(9):829-42.

2. Kim J H, Lee H-K, Takamiya K, Huganir R L. The role of synaptic GTPase-activating protein in neuronal development and synaptic plasticity. J Neurosci. 2003; 23(4):1119-24.

1. A method of treating a subject having a SYNGAP1 neurodevelopmental disorder, comprising:
administering to the subject an effective amount of perampanel.

2. The method of claim 1 wherein the subject is identified as suffering from or susceptible to a sleep disorder and the perampanel is administered to the identified subject.

3. The method of claim 1 wherein the subject is identified as suffering from or susceptible to a behavioral problem and the perampanel is administered to the identified subject.

4. The method 1 through 3 wherein the subject is identified as suffering from or susceptible a myoclonic or reflex seizure and the perampanel is administered to the identified subject.

5. A method of treating a subject having a SYNGAP1 neurodevelopmental disorder, comprising:
identifying a subject as suffering from a SYNGAP1 neurodevelopmental disorder;
selecting the identified subject for treatment; and
administering to the identified and selected subject an effective amount of perampanel, or,
A method of treating a subject having a sleep disorder, comprising:
identifying a subject as suffering from a sleep disorder;
selecting the identified subject for treatment; and
administering to the identified and selected subject an effective amount of perampanel. or,
A method of treating a subject having a behavioral problem, comprising:
identifying a subject as suffering from a behavioral problem;
selecting the identified subject for treatment; and
administering to the identified and selected subject an effective amount of perampanel, or,
A method of treating a subject suffering or susceptible to myoclonic or reflex seizures, comprising:
identifying a subject as suffering or susceptible to myoclonic or reflex seizures;
selecting the identified subject for treatment; and
administering to the identified and selected subject an effective amount of perampanel.

6-11. (canceled)

12. The method of claim 1 wherein up to 0.5 mg of perampanel is administered to the subject per day.

13. The method of claim 1 wherein up to 1.0 mg of perampanel is administered to the subject per day.

14. The method of claim 1 wherein up to 1.5 mg of perampanel is administered to the subject per day.

15. The method of claim 1 wherein up to 2.0 mg of perampanel is administered to the subject per day.

16-27. (canceled)

28. The method of claim 1 wherein the subject is a pediatric patient.

29-32. (canceled)

33. The method of claim 1 wherein the subject is a human subject.

34-37. (canceled)

38. A pharmaceutical composition comprising perampanel in an amount of less than 2 mg in a unit dosage oral form, or
A kit comprising:
(a) perampanel;
(b) instructions for use of the perampanel for treatment of a condition or disorder associated with a SYNGAP1 neurodevelopmental disorder, or,
A kit comprising:
(a) perampanel:
(b) instructions for use of the perampanel for treatment of a sleep disorder, or,
A kit comprising:
(a) perampanel:
(b) instructions for use of the perampanel for treatment of a behavioral problem, or,
A kit comprising:
(a) perampanel;
(b) instructions for use of the perampanel for treatment of a myoclonic or reflex seizure.

39-46. (canceled)

47. A method of claim 1 comprising identifying a subject as suffering from a SYNGAP1 neurodevelopmental disorder; selecting the identified subject for treatment; and administering to the identified and selected subject the effective amount of perampanel.

48. The method of claim 47 wherein up to 2.0 mg of perampanel is administered to the subject per day.

49. The method of claim 1 comprising identifying a subject as suffering from a sleep disorder; selecting the identified subject for treatment; and administering to the identified and selected subject the effective amount of perampanel,

50. The method of claim 49 wherein up to 2.0 mg of perampanel is administered to the subject per day.

51. The method of claim 1 comprising identifying a subject as suffering from a behavioral problem; selecting the identified subject for treatment; and administering to the identified and selected subject the effective amount of perampanel,

52. The method of claim 51 wherein up to 2.0 mg of perampanel is administered to the subject per day.

53. The method of claim 1 comprising identifying a subject as suffering or susceptible to myoclonic or reflex seizures; selecting the identified subject for treatment; and administering to the identified and selected subject an effective amount of perampanel.

54. The method of claim 53 wherein up to 2.0 mg of perampanel is administered to the subject per day.
